(19) United States
(12) Patent Application Publication
(10) Pub. No.: US 2010/0152238 A1
(43) Pub. Date: Jun. 17, 2010

(54) NOVEL QUINONOID DERIVATIVES OF CANNABINOIDS AND THEIR USE IN THE TREATMENT OF MALIGNANCIES

(75) Inventors: Raphael Mechoulam, Jerusalem (IL); Natalya Kogan, Jerusalem (IL)

Correspondence Address: THE NATH LAW GROUP, 112 South West Street, Alexandria, VA, 22314, US

(73) Assignee: Yissum Research Development Company of The Hebrew University of Jerusalem, Jerusalem (IL)

(21) Appl. No.: 12/449,980

(22) PCT Filed: Mar. 5, 2008
(86) PCT No.: PCT/IL2008/000280

§ 371 Date: Feb. 23, 2010

Related U.S. Application Data
(60) Provisional application No. 60/904,806, filed on Mar. 5, 2007.
(60) Provisional application No. 60/990,364, filed on Nov. 27, 2007.

(51) Int. Cl. A61K 31/451; C07C 69/734; C07C 61/35; C07C 49/753; C07D 211/32; C07C 211/25; A61K 31/19; A61K 31/13; A61K 31/122; A61P 35/00; A61P 35/02; A61P 17/06; A61P 17/00; A61P 1/00; A61P 11/00; A61K 31/215

(52) U.S. Cl. 514/317; 560/118; 562/500; 568/367; 546/237; 564/453; 514/557; 514/660; 514/691; 514/529

(57) ABSTRACT

Novel cannabinoid-derived quinone derivatives (quinonoid derivatives) having a substituted hydroxyl group, pharmaceutical compositions comprising same and uses thereof as anti-proliferative agents, are provided.

### FIELD AND BACKGROUND OF THE INVENTION

The present invention, in some embodiments thereof, relates to novel quinonoid derivatives of cannabinoids, also referred to herein interchangeably as cannabinoid quinones, to pharmaceutical compositions comprising same and to uses thereof as anti-cancerous agents.

Quinones of various chemical families, present in plants and animals, serve as biological modulators and both natural and synthetic quinones are widely used as drugs.

Anthracyclines, a large group of quinonoid compounds produced by different strains of Streptomyces, exert antibiotic and antineoplasic effects and are used to treat some forms of cancer. The best known members of this family are daunorubicin and doxorubicin, the first identified anthracyclins.

Other quinones are also used as anticancer drugs. Mitomycin C and streptonigrin produced by Streptomyces and the synthetic epirubicin and mitoxantron are well known examples. Although these and other quinonoid compounds are effective in the treatment of many different forms of cancer, their side effects, the most severe of them being cumulative heart toxicity, limit their use. Thus, development of quinonoid compounds that display antineoplastic activity, but are less toxic, is a major therapeutic goal [1-3].

A large number of cannabinoids have been synthesized and tested in the in-vitro and in vivo models of various diseases [4-6]. Cannabinoid-derived quinones were described and studied for a wide range of therapeutic uses and indications [7-14]. WO 2005067917 provides quinonoid derivatives useful as anti-proliferative and anti-inflammatory agents.

### SUMMARY OF THE INVENTION

The present invention, in some embodiments thereof, provides novel cannabinoid-derived quinone derivatives. The present invention further provides pharmaceutical compositions containing these cannabinoid-derived quinone derivatives and uses thereof in the treatment of proliferative diseases and disorders.

Thus, according to one aspect of the present invention there is provided a compound having general Formula I:

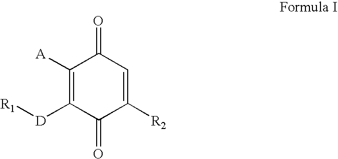

an enantiomer, a hydrate, a solvate or a pharmaceutically acceptable salt thereof;

wherein:

A is selected from the group consisting of an unsubstituted or substituted cycloalkyl, an unsubstituted or substituted heteroalicyclic, an unsubstituted or to substituted aryl and an unsubstituted or substituted heteroaryl;

R₁ is selected from the group consisting of hydrogen and an unsubstituted or substituted, branched or linear alkyl having from 1 to 10 carbon atoms;

R₂ is selected from the group consisting of an unsubstituted or substituted, branched or linear alkyl having from 1 to 10 carbon atoms, an alkoxy and an aryloxy;

D is selected from the group consisting of NR₃, O and S; and

R₃ is an unsubstituted or substituted, branched or linear alkyl having from 1 to 10 carbon atoms.

According to further features in some embodiments of the invention described herein D is O.

According to still further features in some embodiments of the invention described herein, A is selected from the group consisting of an unsubstituted or substituted cycloalkyl, an unsubstituted or substituted heteroalicyclic and an unsubstituted or substituted heteroaryl.

According to still further features in some embodiments of the invention described herein, A is an unsubstituted or substituted heteroalicyclic.

According to still further features in some embodiments of the invention described herein, R₂ is selected from the group consisting of pentyl and dimethyl-heptyl.

According to still further features in some embodiments, R₁ is hydrogen, and A is 1-methylpiperidin-4-yl.

According to still further features in some embodiments, A is an unsubstituted or substituted cycloalkyl.

According to still further features in some embodiments, the cycloalkyl is selected from the group consisting of a monocyclic unsubstituted or substituted cycloalkyl and a bicyclic unsubstituted or substituted cycloalkyl.

According to yet further features in some embodiments, the bicyclic unsubstituted or substituted cycloalkyl is an unsubstituted or substituted pinene.

According to yet further features in some embodiments, the monocyclic unsubstituted or substituted cycloalkyl has general Formula II:

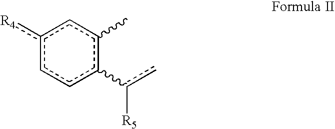

wherein:

R₄ and R₅ are each independently selected from the group consisting of hydrogen, alkyl, haloalkyl, halo, hydroxyl, alkoxy, carboxyl, carbonyl, formyl, acetyl and amine;

whereas:

a dashed line is a single or double bond; and

a wavy line is a bond having an R or an S stereo-configuration.

According to yet further features in some embodiments, R₁ is selected from the group consisting of an unsubstituted or substituted, branched or linear alkyl having from 6 to 10 carbon atoms and a substituted, branched or linear alkyl having from 1 to 10 carbon atoms.

According to other features in some embodiments, R₁ is a substituted, branched or linear alkyl having from 1 to 5 carbon atoms.

According to other features in some embodiments, A is 3-methyl-6-(prop-1-en-2-yl)cyclohex-2-enyl.

According to other features in some embodiments, R₁ is selected from the group consisting of 2-yl-acetic acid, ethyl 2-yl-acetate, ethoxy-2-oxo-ethane-1-yl, ethanol-2-yl, ethanamine-2-yl, N-Boc-ethanamine-2-yl, N-Fmoc-ethanamine-2-yl, 3-morpholinopropanoyl and acetonitrile-2-yl.

According to other features in some embodiments, R₁ is selected from the group consisting of ethoxy-2-oxo-ethane-1-yl, 2-yl-acetic acid, ethanol-2-yl and ethanamine-2-yl.

According to other features in some embodiments, R₂ is selected from the group consisting of pentyl and dimethyl-heptyl.

According to other features in some embodiments, R₂ is 1-pentyl.

According to other features in some embodiments, R₂ is selected from the group consisting of pentyl and dimethyl-heptyl.

According to another aspect of the present invention there are provided compounds selected from the group consisting of:
ethyl 2-(2-((6R)-3-methyl-6-(prop-1-en-2-yl)cyclohex-2-enyl)-3,6-dioxo-5-pentylcyclohexa-1,4-dienyloxy)acetate (HU-701);
2-(2-((6R)-3-methyl-6-(prop-1-en-2-yl)cyclohex-2-enyl)-3,6-dioxo-5-pentylcyclohexa-1,4-dienyloxy)acetic acid (HU-702);
3-(2-hydroxyethoxy)-2-((6R)-3-methyl-6-(prop-1-en-2-yl)cyclohex-2-enyl)-5-pentylcyclohexa-2,5-diene-1,4-dione (HU-703);
3-(2-aminoethoxy)-2-((6R)-3-methyl-6-(prop-1-en-2-yl)cyclohex-2-enyl)-5-pentylcyclohexa-2,5-diene-1,4-dione (HU-704);
3-hydroxy-2-(1-methylpiperidin-4-yl)-5-pentylcyclohexa-2,5-diene-1,4-dione (HU-705);

and any enantiomer, hydrate, solvate or pharmaceutically acceptable salt thereof.

According to features in some embodiments of the present invention, the compounds presented herein exhibit anti-proliferative activity.

Thus, according to another aspect of the present invention there is provided a pharmaceutical composition which includes, as an active ingredient, a compound as presented herein.

According to features in some embodiments of the present invention, the pharmaceutical composition is being packaged in a packaging material and identified in print, in or on the packaging material, for use in the treatment of a proliferative disease or disorder.

According to another aspect of the present invention there is provided a method of treating a proliferative disease or disorder, which is effected by administering to a patient in need thereof a therapeutically effective amount of a compound as presented herein.

According to another aspect of the present invention there is provided a use of a compound as presented herein, in the preparation of a medicament

According to features in some embodiments, the medicament is for the treatment of a proliferative disease or disorder.

According to features in some embodiments of the present invention, the proliferative disease or disorder is selected from the group consisting of a malignant proliferative disease or disorder, a non-malignant proliferative disease or disorder, an inherent proliferative disease or disorder and an acquired proliferative disease or disorder.

According to features in some embodiments of the present invention, the malignant proliferative disease or disorder is selected from the group consisting of blastoma, carcinoma, lymphoma, leukemia, sarcoma, mesothelioma, glioma, germinoma, choriocarcinoma, melanoma, glioblastoma, lymphoid malignancy and any other neoplastic (cancerous) disease or disorder.

According to features in some embodiments of the present invention, the non-malignant proliferative disease or disorder is selected from the group consisting of psoriasis, endometriosis, scleroderma, a vascular disease, colon polyps, fibroadenoma and a respiratory disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a,” “an,” and “the” include plural references unless the context clearly dictates otherwise. For example, the term “a compound” or to “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein throughout the term “about” refers to ±10%.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents the results of an in-vitro cell proliferation assay on Jurkat human lymphoma cell line, conducted using exemplary compounds according to some embodiments of the present invention, HU-701, HU-702, HU-703, HU-704 and HU-705, and compared to HU-331.

FIG. 2 presents the results of an in-vitro cell proliferation assay of human colon carcinoma HT-29 cell line, using exemplary compounds according to some embodiments of the present invention, HU-701, HU-702, HU-703, HU-704 and HU-705, and compared to HU-331.

### DESCRIPTION OF THE PREFERRED EMBODIMENTS

The present invention, in some embodiments thereof, provides novel cannabinoid-derived quinone derivative compounds, also referred to herein interchangeably as quinonoid derivatives. The present invention further provides pharmaceutical compositions containing these quinonoid derivatives and uses thereof in the treatment of proliferative diseases and disorders.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As discussed hereinabove, previous studies of cannabinoid-derived quinone derivative compounds were prepared and evaluated as potential anti-cancerous agents ([8, 3, 14-16] and WO 2005067917). Some embodiments of the present invention provide novel cannabinoid-derived quinone derivative compounds, also referred to herein interchangeably as quinonoid derivatives or cannabinoid-derived quinones, showing improved anti-proliferative activity.

While conceiving the present invention, it was found that by introducing some types of substituents on a hydroxyl group on the central aromatic ring of some cannabinoid derivatives, more active compounds are afforded, having improved solubility and thus present a significant improvement of their therapeutic value.

While reducing the present invention to practice, the novel cannabinoid-derived quinones disclosed herein were evaluated as medicinal anti-proliferative agents. Hence, the present invention encompasses the medicinal use of these quinone derivatives, especially with regards to their potent anti-neoplastic and anti-cancerous activity in vitro and in-vivo.

Thus, according to one aspect of the present invention, there is provided a compound having general Formula I:

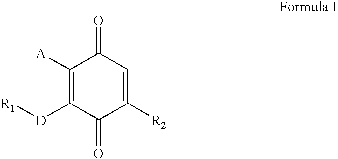

wherein:

A is selected from the group consisting of an unsubstituted or substituted cycloalkyl, an unsubstituted or substituted heteroalicyclic, an unsubstituted or substituted aryl and an unsubstituted or substituted heteroaryl;

R₁ is selected from the group consisting of hydrogen and an unsubstituted or substituted, branched or linear alkyl having from 1 to 10 carbon atoms;

R₂ is selected from the group consisting of an unsubstituted or substituted, branched or linear alkyl having from 1 to 10 carbon atoms, an alkoxy and an aryloxy;

D is selected from the group consisting of NR₃, O and S; and

R₃ is an unsubstituted or substituted, branched or linear alkyl having from 1 to 10 carbon atoms.

The present embodiments further encompass any enantiomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound as presented herein, having one or more carboxylic acid moieties, which is administered as an ester, or amine group which is administered as an amide (the “prodrug”). Such a prodrug is hydrolyzed in vivo, to thereby provide the free compound (the parent drug). The selected ester may affect both the solubility characteristics and the hydrolysis rate of the prodrug. Prodrugs, according to some embodiments of the present invention, can be made using succinic acid, maleic acids, fumaric acids and the likes.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. An example, without limitation, of a pharmaceutically acceptable salt would be a carboxylate anion and a cation such as, but not limited to, ammonium, sodium, potassium and the like.

For example, a compound in which D is O, R₁ is an alkyl substituted with a carboxyl group (see for example, HU-702, in the Examples section that follows below) exhibits an improved bioavailability profile by being highly soluble in aqueous media in concentrations much higher than those of the previously known quinonoid derivatives, Furthermore, such a quinonoid derivative can be readily converted into an anion of many pharmaceutically acceptable salts having cations such as, for example, sodium, potassium, ethylenediamine, ethanolamine, calcium, deanol, magnesium, zinc, piperazine, diethanolamine, pyrrolidine, betaine, tromethamine, choline, lysine, morpholine, triethanolamine, arginine, N-methylglucamine and the likes. Alternatively, such a derivative can be readily turned into an ester, such as the ethyl ester HU-701 (see, Table 1 herebelow), and be administered as a prodrug of compound HU-702.

In another example, a compound in which D is N (nitrogen), namely a secondary or a tertiary amine, the parent compound can be ionized so as to be positively charged and hence be a cation of a salt.

In yet another example, a compound which contains an amine group on either one of R₁ or A, can be converted to a cation of a pharmaceutically acceptable acid addition salt.

As is well known in the art, the phrase “acid addition salt” describes a complex of two ionizable moieties, a base and an acid, which, when interacted in a particular stoichiometric proportion and under suitable conditions, form a salt that comprises one or more cations of the base moiety and one or more anions of the acid moiety. As used herein, the phrase “acid addition salt” refers to such a complex, in which the base moiety in amine, such that the salt comprises a cationic form of the amine (ammonium) and an anionic form of an acid.

Depending on the stoichiometric proportions between the base and the acid in the salt complex, as is detailed hereinbelow, the acid additions salts can be either mono addition salts or poly addition salts.

The phrase “mono addition salt”, as used herein, refers to a salt complex in which the stoichiometric ratio between the acid anion and amine cation is 1:1, such that the acid addition salt includes one molar equivalent of the acid per one molar equivalent of the conjugate.

The phrase “poly addition salt”, as used herein, refers to a salt complex in which the stoichiometric ratio between the acid anion and the amine cation is greater than 1:1 and is, for example, 2:1, 3:1, 4:1 and so on, such that the acid addition salt includes two or more molar equivalents of the acid per one molar equivalent of the conjugate.

The stoichiometric proportions between the base and the acid of the salt complex, according to some embodiments of the present invention, ranges from 6:1 to 1:6 base:acid equivalents, from 4:1 to 1:4 base:acid equivalents, from 3:1 to 1:3 base:acid equivalents or from 1:1 to 1:3 base:acid equivalents.

The acid addition salts of a chemical conjugate according to the present invention are therefore complexes formed between one or more amino groups of the compound and one or more equivalents of an acid. The acid addition salts may therefore include a variety of organic and inorganic acids, such as, but not limited to, halogen acids such as hydrochloric acid which affords an hydrochloric acid addition salt (as well as salts of bromide and iodide), acetic acid which affords an acetic acid addition salt, ascorbic acid which affords an ascorbic acid addition salt, benzoic acid which affords a benzoic acid addition salt (benzoate), benzenesulfonic acid which affords a benzenesulfonic acid addition salt, camphorsulfonic acid which affords a camphorsulfonic acid addition salt, naphthylsulfonic acid which affords a naphthylsulfonic acid addition salt, toluenelsulfonic acid (p-toluenesulfonic acid) which affords a toluenesulfonic acid addition salt (tosylate), trifluoroacetic acid which affords a trifluoroacetic acid addition salt, citric acid which affords a citric acid addition salt, maleic acid which affords a maleic acid addition salt (maleate), methanesulfonic acid which affords a methanesulfonic acid (mesylate or methanesulfonate) addition salt, naphthalenesulfonic acid which affords a napsylate addition salt, oxalic acid which affords an oxalic acid addition salt, phosphoric acid which affords a phosphoric acid addition salt, succinic acid which affords a succinic acid addition salt (succinate), sulfuric acid which affords a sulfuric acid addition salt and tartaric acid which affords a tartaric acid addition salt. Each of these acid addition salts can be either a mono acid addition slat or a poly acid addition salt, as these terms are defined hereinabove.

In another example, quinonoid derivative compounds, as presented herein, which contain one or more —OH (hydroxyl) or an —NH₂ (amine) groups either on one of R₁ or A, can be converted to into a prodrug by coupling to, for example, a succinic, fumaric, maleic acids and other suitable acids to form prodrugs, which can be enzymatically hydrolyzed in the body by, for example, esterases or amidases.

All the quinonoid derivative compounds presented herein (namely ethers, esters, salts, prodrugs, etc.) are considerably more soluble in aqueous media as compared to the previously described quinonoid derivatives.

As described hereinabove, A is a cyclic moiety which can be saturated, partly saturated or aromatic (cycloalkyl or aryl), which can have one or more heteroatom as part of the ring (heteroalicyclic or heteroaryl), and further be substituted and substituted.

The term “cycloalkyl” (also known as alicyclic), as used herein, describes an all-carbon monocyclic or fused ring (i.e., rings which share an adjacent pair of carbon atoms) group where one or more of the rings does not have a completely conjugated pi-electron system. The cycloalkyl may be unsubstituted or substituted by one or more substituents. When substituted, the substituent can be, for example, an alkyl, an alkenyl, an alkynyl, a cycloalkyl, an aryl, a heteroaryl, a halogen (halo), a hydroxy, an alkoxy, an aryloxy, a thiohydroxy, a thioalkoxy, a thioaryloxy, a haloalkyl, an amine, a carbonyl, a carboxyl, an amide, a thioamide, a cyano and a carbamate, as well as combinations thereof, as these terms are defined herein.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be unsubstituted or substituted by one or more substituents, as described hereinabove for cycloalkyl. Representative examples of heteroalicyclics include, without limitation, piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be unsubstituted or substituted by one or more substituents, as described hereinabove for cycloalkyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. The heteroaryl group may be unsubstituted or substituted by one or more substituents, as described hereinabove for cycloalkyl. Examples, without limitation, of heteroaryl groups include pyrrole, furane, thiophene, imidazole, oxazole, thiazole, pyrazole, pyridine, pyrimidine, quinoline, isoquinoline and purine.

A representative group of moieties which can embody A in Formula I include, according to some embodiments of the present invention and without limitation, [1,2]diazocan-3-one, [1,3]diazocan-2-one, [1,4]diazocane, [1,4]oxazepane, 1,2,3-triazine, 1,2,3-triazole, 1,2,4-triazine, 1,2,4-triazole, 1,2-diazepine, 1,2-oxathiepane, 1,2-oxathiolane, 1,2-oxazine, 1,2-thiazine, 1,3,5-triazine, 1,3-diazepine, 1,3-dioxolane, 1,3-dioxolene, 1,3-oxazine, 1,3-thiazine, 1,3-thiazole, 1,4-diazapane, 1,4-diazepine, 1,4-oxazepane, 1,4-oxazine, 1,4-thiazine, 2-isoxazoline, 5,6,7,8-tetrahydro-1H-azocin-2-one, acridine, azaspirodecan, azepine, azetidine, aziridine, azirine, azocane, azocane-2-one, benzimidazole, benzofuran, benzothiazole, benzothiophene, benzoxazole, carbazole, cinnoline, cyclohexyl, diaziridine, diazirine, dioxane, dioxazine, dioxazole, dioxin, dioxolane, dioxole, dithiane, dithiazine, dithiazole, dithiolane, furan, imidazole, imidazolidine, imidazoline, indazole, indole, indoline, indolizine, isoindole, isoquinoline, isothiazole, isothiazolidine, isothiazoline, isoxazole, isoxazolidine, isoxazoline, ketopiperazine, morpholine, napthyridine, oxadiazine, oxadiazole, oxathiazole, oxathiazolidine, oxazine, oxaziridine, oxazole, oxirane, oxocan-2-one, oxocane, phenazine, phenothiazine, phenoxazine, phenyl, phthalazine, piperazine, piperidine, pteridine, purine, pyran, pyrazine, pyrazole, pyrazolidine, pyrazoline, pyrazoline, pyridazine, pyridine, pyrimidine, pyrrole, pyrrolidine, pyrrolidinone, pyrroline, quinazoline, quinoline, quinolizine, quinoxaline, tetrahydrofuran, tetrahydropyridine, tetrazine, tetrazole, thiadiazine, thiadiazole, thianaphthalene, thiatriazole, thiazine, thiazole, thiazolidine, thiazoline, thienyl, thietan, thiomorpholine, thiophene, thiopyran, triazine, triazole and trithiane.

Compounds having general Formula I wherein A is a cycloalkyl or an aryl moiety, D is O (oxygen), and R₁ is hydrogen or an unsubstituted branched or linear alkyl having from 1 to 5 carbon atoms, have been previously described (for example, in WO 2005067917) and are therefore excluded from the scope of this aspect of the present invention.

As used herein, the term “alkyl” describes an aliphatic hydrocarbon including straight chain and branched chain groups. According to some embodiments, and unless specified otherwise, an alkyl group has 1 to 10 carbon atoms; according to other embodiments 1 to 5 carbon atoms, according to yet other embodiments 6 to 10 carbon atoms; and according to still other embodiments 4 to 6 carbon atoms. Whenever a numerical range; e.g., “1 to 10”, is stated herein, it implies that the group, in this case the alkyl group, may contain 1 carbon atom, 2 carbon atoms, 3 carbon atoms, etc., up to and including 10 carbon atoms. The alkyl can be unsubstituted or substituted. When substituted, the substituent can be, for example, an alkyl, an alkenyl, an alkynyl, a cycloalkyl, an aryl, a heteroaryl, a halogen (halo), a hydroxy, an oxo, an alkoxy, an aryloxy, a thiohydroxy, a thioalkoxy, a thioaryloxy, a haloalkyl, an amine, a carbonyl, a carboxyl, an amide, a thioamide, a cyano and a carbamate, as these terms are defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be unsubstituted or substituted by one or more substituents, as described hereinabove for alkyl.

The term “alkynyl”, as defined herein, is an unsaturated alkyl having at least two carbon atoms and at least one carbon-carbon triple bond. The alkynyl may be unsubstituted or substituted by one or more substituents, as described hereinabove for alkyl.

As used herein, the term “amine” describes a —NR′R″ group where each of R′ and R″ is independently hydrogen, alkyl, cycloalkyl, heteroalicyclic, aryl or heteroaryl, as these terms are defined herein.

As used herein, the terms “halo”, “halogen” and “halide”, which are referred to herein interchangeably, describe an atom of a fluorine, chlorine, bromine or iodine, also referred to herein as fluoride, chloride, bromide and iodide.

The term “haloalkyl” describes an alkyl group as defined above, further substituted by one or more halide(s).

The term “hydroxy” or “hydroxyl”, as used herein interchangeably, refers to an —OH group.

The term “alkoxy” describes a —OR′ group, where R′ is as defined herein.

The term “aryloxy”, as used herein, refers to an —OR″ group wherein R″ is aryl.

The term “thiohydroxy”, as used herein, refers to an —SH group.

The term “thioalkoxy” describes a —SR′ group, where R′ is as defined herein.

The term “thioarylkoxy” describes a —SR″ group, where R″ is aryl.

The term “carbonyl”, or “ketone”, as used herein, refers to —(C═O)H or —(C═O)—R′ group, wherein R′ is as defined herein. An exemplary carbonyl is a formyl group, wherein R′ is hydrogen. Another exemplary carbonyl is an acetyl group, wherein R′ is methyl.

The term “oxo” refers to a (═O) group, namely an oxygen bound by a double bond, which in the case of a carbon substituent constitutes a carbonyl.

The terms “carboxy”, “carboxyl” or “carboxylate”, as used herein, refer interchangeably to a —C(═O)—O—R′, where R′ can be absent (as in the case of a carboxylate anion), or selected from the group consisting of hydrogen (for example, carboxylic acid), alkyl (for example, ester), cycloalkyl, heteroalicyclic, aryl or heteroaryl, as these terms are defined herein.

The term “amide” describes a —C(═O)—NR′R″, where R′ is as defined herein and R″ is as defined for R′.

The term “thioamide” describes a —C(═S)—NR′R″, where R′ is as defined herein and R″ is as defined for R′.

The term “thioimide” describes a —C(═NR′)—SR″, where R′ and R″ are as defined herein.

The term “cyano”, as used herein, refers to a —C≡N group. For example an acetonitrile substituent group is a cyano group attached to a molecule via a —CH₂— group, constituting a —CH₂—C≡N group.

The term “carbamate” describes an —OC(═O)—NR′R″, with R′ and R″ as defined herein.

A particular exemplary carbamate is afforded when an amine is protected with a Boc protecting group, affording a tert-butyl carbamate.

Another exemplary carbamate is afforded when an amine is protected with an Fmoc protecting group, affording a (9H-fluoren-9-yl)methyl carbamate.

It is therefore noted that unless stated otherwise, R₁ and/or R₂ can each be unsubstituted or substituted with a number of groups as presented hereinabove, as well as combinations thereof, and the same definition applies to any variable which is defined as unsubstituted or substituted, regardless of the definition for each of the particular chemical groups.

As presented in Formula I, R₂ can be an unsubstituted or substituted, branched or linear alkyl having from 1 to 10 carbon atoms, an alkoxy or an aryloxy. In many cannabinoids and in compounds according to some embodiments of the present invention, the group which is equivalent to the R₂ variable in Formula I is 1-pentyl or dimethylheptyl (DMH). In some exemplary cannabinoids and in compounds according to some embodiments of the present invention, this group can be, for example, 1,2-dimethylheptyl, 1,1-dimethylheptyl and the likes.

Optionally, R₂ can be an alkyl having from 1 to 10 carbon atoms, which is terminated on one or both ends thereof, or interrupted by one or more oxygen, nitrogen or sulfur atoms, and further can be optionally terminated with an alkoxy group or an aryloxy group, as these are defined herein.

In some embodiments, R₂ can be a straight (linear) or branched alkyl of 5 to 12 carbon atoms; a group —O-alkyl, where the alkyl is straight (linear) or branched having 5 to 9 carbon atoms, or a straight (linear) or branched alkyl substituted at the terminal carbon atom by a phenyl group; a group —(CH₂)n—O-alkyl, where n is an integer from 1 to 7 and the alkyl group contains 1 to 5 carbon atoms.

As mentioned hereinabove, in some embodiments of the present invention, A can be an unsubstituted or substituted heteroalicyclic, as defined and exemplified above. In such cases, and according to some embodiments, R₁ is hydrogen or an unsubstituted or substituted, branched or linear alkyl having from 1 to 10 carbon atoms. One exemplary compound which belongs to this particular group of compounds, is compound HU-705 (see, Table 1 below), wherein R₁ is hydrogen, and A is 1-methylpiperidin-4-yl.

According to other embodiments of the present invention, the novel compounds presented herein are derivatives of cannabidiol (CBD), hence A can be a monocyclic unsubstituted or substituted cycloalkyl and a bicyclic unsubstituted or substituted cycloalkyl.

In some of these embodiments, A can be a substituted monocyclic six-membered cycloalkyl.

In yet other embodiments, the substituted monocyclic six-membered cycloalkyl is a moiety having general Formula II:

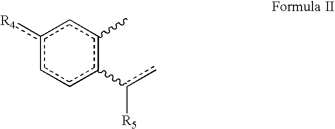

wherein:

R₄ and R₅ are each independently selected from the group consisting of hydrogen, alkyl, haloalkyl, halo, hydroxyl, alkoxy, carboxyl, carbonyl, formyl, acetyl and amine, as there terms are defined hereinabove.

Each of the dashed lines in Formula II represents a single or double bond, and each of the wavy lines represents a bond having an R or an S stereo-configuration.

It is noted herein that each of R₄ and R₅, independently, can be attached to main part of the moiety having general Formula II via a single bond or a double bond, depending on the nature thereof and the valency of the atom these groups are attached to.

It is further noted that the feasibility of each of the variables in Formula I and Formula II, namely A, D and R₁—R₅, the bonds variables located at the indicated positions as well as their optional substituents, depends on the valency and chemical compatibility of the participating variable, bond or substituent, the substituted position and other neighboring substituents. Hence, compounds represented by general formulae according to some embodiments of the present invention, are aimed at encompassing only the chemically feasible molecules, having only the chemically feasible substituents for any given position.

Compounds wherein A has general Formula II, D is oxygen, and R₁ is a substituted or unsubstituted alky having 1-5 carbon atoms have been described previously in WO 2005067917, and are therefore excluded from the scope of the present embodiments. However, compounds wherein D is not oxygen, or wherein A is a substituted or unsubstituted cycloalkyl which does not have the general Formula II, or wherein R₁ is a substituted or unsubstituted alkyl having more than 5 carbon atoms are encompassed by some of the present embodiments.

According to some embodiments of the present invention, when D is O, and A is a moiety having general Formula II, the compounds according to some embodiments of the present invention share many structural features of the naturally occurring CBD molecule. In order to improve the pharmaceutical profile and yet maintain a viable and scaleable synthesis, these compounds were designed and selected such that their preparation and their bioavailability are improved by virtue of the particular substituents at any of variables R₁—R₅, and in addition when the resulting compound can be ionized at physiological pH, namely an acid or a base that can be turned into a salt thereof.

Alternatively, D is O, and A is 3-methyl-6-(prop-1-en-2-yl)cyclohex-2-enyl, as in CBD, while R₁ can be a substituted, branched or linear alkyl having from 1 to 10 carbon atoms; an unsubstituted or substituted alkyl having 6 to 10 carbon atoms; or an unsubstituted or substituted alkyl which is terminated on one or both ends thereof, or interrupted by one or more oxygen, nitrogen or sulfur atoms.

In some embodiments of the present invention, R₁ is a substituted linear short alkyl, having, for example, 1 to 5 carbon atoms. The alkyl is substituted by, for example, one or more of oxo, hydroxy, carboxy, amine and nitrile.

Hence, according to some embodiments of the present invention, R₁ can be, without limitation, 2-yl-acetic acid, ethyl 2-yl-acetate, ethoxy-2-oxo-ethane-1-yl, ethanol-2-yl, ethanamine-2-yl, N-Boc-ethanamine-2-yl, N-Fmoc-ethanamine-2-yl, 3-morpholinopropanoyl and acetonitrile-2-yl.

According to other embodiments, R₁ is selected from According to other features in some embodiments, R₁ is selected from the group consisting of ethoxy-2-oxo-ethane-1-yl, 2-yl-acetic acid, ethanol-2-yl and ethanamine-2-yl.

Further, when the variable R₂ is 1-pentyl, and according to embodiments of the present invention, these quinonoid derivative compounds are part of a group of compounds which include, without limitation, ethyl 2-(2-((6R)-3-methyl-6-(prop-1-en-2-yl)cyclohex-2-enyl)-3,6-dioxo-5-pentylcyclohexa-1,4-dienyloxy)acetate (HU-701); 2-(2-((6R)-3-methyl-6-(prop-1-en-2-yl)cyclohex-2-enyl)-3,6-dioxo-5-pentylcyclohexa-1,4-dienyloxy)acetic acid (HU-702); 3-(2-hydroxyethoxy)-2-((6R)-3-methyl-6-(prop-1-en-2-yl)cyclohex-2-enyl)-5-pentylcyclohexa-2,5-diene-1,4-dione (HU-703); 3-(2-aminoethoxy)-2-((6R)-3-methyl-6-(prop-1-en-2-yl)cyclohex-2-enyl)-5-pentylcyclohexa-2,5-diene-1,4-dione (HU-704); and 3-hydroxy-2-(1-methylpiperidin-4-yl)-5-pentylcyclohexa-2,5-diene-1,4-dione (HU-705), all of which are presented in Table 1 in the Examples section that follows.

As many of the presently known and widely studied cannabinoids are plant derived, a large family thereof contains a pinene moiety, which is one of the more ubiquitous natural transformation of 1-methyl-4-(prop-1-en-2-yl)cyclohex-1-ene moiety, as found in some of the compounds according to some embodiments of the present invention, particularly those wherein A is having general Formula II. Briefly, the chemical compound pinene is a bicyclic terpene, also known as a monoterpene, which is found both in the α-pinene configuration and the β-pinene configuration (systematic names are (1S,5S)-2,6,6-trimethylbicyclo[3.1.1] hept-2-ene and (1S,5S)-6,6-dimethyl-2-methylenebicyclo[3.1.1]heptane, respectively), which can be metabolized or synthetically produced from, for example, a 1-methyl-4-(isopropen-2-yl)cyclohexene carbocation intermediate, as illustrated in Scheme 1 below.

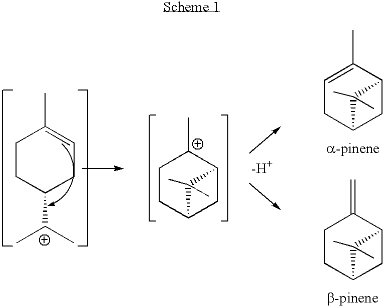

Compounds corresponding to Formula I wherein A is a substituted or unsubstituted pinene have not been described previously and are therefore encompassed by some of the present embodiments.

As described and demonstrated in the Examples section that follows, the compounds according to some embodiments of the present invention have been tested for their anti-proliferative activity, and were indeed found to be highly potent candidates for anti-cancerous agents and drugs.

As used herein, the term “anti-cancerous” as in the phrase “anti-cancerous activity”, refers to a therapeutic activity of a substance which can be used to treat cancer by directly or indirectly inhibiting the growth of neoplastic cells and tissues selectively with respect to benign cells and tissues.

The phrase “neoplastic tissue” as used herein, refers to an abnormal, disorganized and typically uncontrolled proliferation and growth of cells in a tissue or an organ, usually forming a distinct mass of cells which is commonly referred to as a malignant growth, neoplasm or tumor, and collectively referred to as cancer.

Thus, according to another aspect of the present invention, there is provided a method of treating a proliferative disease or a disorder which is effected by administering to a subject in need thereof a therapeutically effective amount of one or more of the compounds as presented herein, as well enantiomers, hydrates, solvates, prodrugs or any pharmaceutically acceptable salts thereof, as defined hereinabove.

As used herein, the phrase “proliferative disease or a disorder” describes an abnormal and thus undesired physiological condition in mammals that is typically characterized by unregulated and oftentimes aggressive cell growth and/or division, which occurs without respect to normal cell or tissue limits. Some proliferative diseases are also characterized by invasive cell growth and/or division, which invade and destroy adjacent tissues, and/or sometimes metastatic proliferation, which spreads to other locations in the body.

According to some embodiments of the present invention, there is provided a method of treating a proliferative disease or a disorder which is effected by administering to a subject in need thereof a therapeutically effective amount of one or more of any one of the compounds presented in Table 1 which is presented in the Examples section that follows hereinbelow, as well as enantiomers, hydrates, solvates, prodrugs or any pharmaceutically acceptable salts thereof, as defined hereinabove.

As used herein, the terms “treating” and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

Accordingly, another aspect of the present invention provides a use of one or more of the compounds as presented herein, as well enantiomers, hydrates, solvates, prodrugs or any pharmaceutically acceptable salts thereof, as defined hereinabove, in the preparation of a medicament for the treatment of a proliferative disease or a disorder.

According to some embodiments of the present invention, there is provided a use of one or more of any one of the compounds presented in Table 1 which is presented in the Examples section that follows hereinbelow, as well as any enantiomers, hydrates, solvates, pro drugs or any pharmaceutically acceptable salts thereof, as defined hereinabove, in the preparation of a medicament for the treatment of a proliferative disease or a disorder.

The proliferative disease or disorder can be a malignant proliferative disease or disorder, a non-malignant proliferative disease or disorder, an inherent proliferative disease or disorder or an acquired proliferative disease or disorder.

As used herein, the term “malignant” is a medical term used to describe a severe and progressively worsening disease which potentially poses a mortal threat to the suffering subject. The term malignant is typically used to describe cancer. Thus, malignancy, as in malignant neoplasm, and malignant tumor, are used synonymously with cancer, and also prefix other oncology terms such as malignant ascites, malignant transformation.

When used to fight a malignant proliferative disease or disorder, the anticancerous compounds presented herein can be used to treat a wide spectrum of cancers (neoplasms), such as blastoma, carcinoma, lymphoma, leukemia, sarcoma, mesothelioma, glioma, germinoma, choriocarcinoma, melanoma, glioblastoma, lymphoid malignancies and any other neoplastic disease or disorder, collectively referred to cancer.

Other examples of cancer which can be treated using the compounds according to some embodiments of the present invention include, but are not limited to, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “non-malignant” is a medical term used to describe benign, non-cancerous or non-neoplastic abnormal proliferative growth, which does not pose a direct mortal threat. A malignant tumor may be contrasted with a non-cancerous benign tumor in that a malignancy is not self-limited in its growth, is capable of invading into adjacent tissues, and may be capable of spreading to distant tissues (metastasizing), while a benign tumor has none of those properties.

Hence, in the context of the present invention, a benign proliferative disorder refers to a state in a patient that relates to cell proliferation and which is recognized as abnormal by members of the medical community. An abnormal state is characterized by a level of a property that is statistically different from the level observed in organisms not suffering from the disorder. Cell proliferation refers to growth or extension by multiplication of cells and includes cell division. The rate of cell proliferation may be measured by counting the number of cells produced in a given unit of time. Examples of benign proliferative disorders include psoriasis and polyps.

Other examples of non-malignant (benign) proliferative diseases or disorders include, without limitation, autoimmune disease (e.g. psoriasis, see definition below), endometriosis, scleroderma, restenosis, polyps such as colon polyps, nasal polyps or gastrointestinal polyps, fibroadenoma, respiratory disease (see definition below), cholecystitis, neurofibromatosis; polycystic kidney disease; inflammatory diseases; skin disorders including psoriasis and dermatitis, vascular disease (see definition below), conditions involving abnormal proliferation of vascular epithelial cells, gastrointestinal ulcers, Menetrier's disease, secreting adenomas or protein loss syndrome, renal disorders, angiogenic disorders, ocular disease such as age related macular degeneration, presumed ocular histoplasmosis syndrome, retinal neovascularization stemming from proliferative diabetic retinopathy, retinal vascularization, diabetic retinopathy, or age related macular degeneration, bone associated pathologies such as osteoarthritis, rickets and osteoporosis, damage following a cerebral ischemic event, fibrotic or edemia diseases such as hepatic cirrhosis, lung fibrosis, carcoidosis, throiditis, hyperviscosity syndrome (blood/systemic), Osler Weber-Rendu disease, chronic occlusive pulmonary disease, or edema following burns, trauma, radiation, stroke, hypoxia or ischemia, hypersensitivity reaction of the skin, diabetic retinopathy and diabetic nephropathy, Guillain-Barre syndrome, graft versus host disease or transplant rejection, Paget's disease, bone or joint inflammation, photoaging (e.g. caused by UV radiation of human skin), benign prostatic hypertrophy, certain microbial infections including microbial pathogens selected from adenovirus, hantaviruses, Borrelia burgdorferi, Yersinia and/or Bordetella induced pertussis, thrombus caused platelet aggregation, reproductive conditions such as endometriosis, ovarian hyperstimulation syndrome, preeclampsia, dysfunctional uterine bleeding or menometrorrhagia, synovitis, atheroma, acute and chronic nephropathies (including proliferative glomerulonephritis and diabetes-induced renal disease), eczema; hypertrophic scar formation, endotoxic shock and fungal infection, familial adenomatosis polyposis, neurodedenerative diseases (e.g. Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury, fibrosis of the lung, kidney or liver, T-cell mediated hypersensitivity disease, infantile hypertrophic pyloric stenosis, urinary obstructive syndrome, psoriatic arthritis and Hasimoto's thyroiditis.

A “respiratory disease”, as used herein, involves the respiratory system and includes chronic bronchitis, asthma including acute asthma and allergic asthma, cystic fibrosis, bronchiectasis, allergic or other rhinitis or sinusitis, an alpha 1-antitrypsin or α₁-antitrypsin (A1AT) deficiency, coughs, pulmonary emphysema, pulmonary fibrosis or hyper-reactive airways, chronic obstructive pulmonary disease and chronic obstructive lung disorder.

An “autoimmune disease”, as used herein, refers to a non-malignant disease or disorder arising from and directed against an individual's own tissues. Examples of autoimmune diseases or disorders include, but are not limited to, inflammatory responses such as inflammatory skin diseases including psoriasis and dermatitis (e.g. atopic dermatitis and contact dermatitis), systemic scleroderma and sclerosis, responses associated with inflammatory bowel disease (such as Crohn's disease and ulcerative colitis), respiratory distress syndrome (including adult respiratory distress syndrome; ARDS), dermatitis, meningitis, encephalitis, uveitis, colitis, glomerulonephritis, allergic conditions such as eczema and asthma and other conditions involving infiltration of T cells and chronic inflammatory responses, atherosclerosis, leukocyte adhesion deficiency, rheumatoid arthritis, systemic lupus erythematosus (SLE), diabetes mellitus (e.g. Type I diabetes mellitus or insulin dependent diabetes mellitis), multiple sclerosis, Reynaud's syndrome, autoimmune thyroiditis, allergic encephalomyelitis, Sorgen's syndrome, juvenile onset diabetes, immune responses associated with acute and delayed hypersensitivity mediated by cytokines and T-lymphocytes typically found in tuberculosis, sarcoidosis, polymyositis, granulomatosis and vasculitis, pernicious anemia (Addison's disease), diseases involving leukocyte diapedesis, central nervous system (CNS) inflammatory disorder, multiple organ injury syndrome, hemolytic anemia (including, but not limited to cryoglobinemia or Coombs positive anemia), myasthenia gravis, antigen-antibody complex mediated diseases, anti-glomerular basement membrane disease, antiphospholipid syndrome, allergic neuritis, Grave's disease, Lambert-Eaton myasthenic syndrome, pemphigoid bullous, pemphigus, autoimmune poly-endocrinopathies, Reiter's disease, stiff-man syndrome, Behcet disease, giant cell arteritis, immune complex nephritis, IgA nephropathy, IgM polyneuropathies, immune thrombocyto-penicpurpura (ITP) or autoimmune thrombocytopenia.

The term “psoriasis” as used here, refers to a proliferative medical condition characterized by the eruption of circumscribed, discrete and confluent, reddish, silvery-scaled maculopapules. Psoriatic lesions generally occur predominantly on the elbows, knees, scalp, and trunk, and microscopically show characteristic parakerotosis and elongation of rete ridges. The term includes the various forms of psoriasis, including erythrodermic, pustular, moderate-severe and recalcitrant forms of the disease.

The term “endometriosis” refers to the ectopic occurrence of endometrial tissue, frequently forming cysts containing altered blood.

The phrase “vascular disease or disorder” as used herein, refers to the various diseases or disorders which impact the vascular system, including the cardiovascular system. Examples of such diseases include arteriosclerosis, vascular reobstruction, atherosclerosis, postsurgical vascular stenosis, restenosis, vascular occlusion or carotid obstructive disease, coronary artery disease, angina, small vessel disease, hypercholesterolemia, hypertension, and conditions involving abnormal proliferation or function of vascular epithelial cells.

The term “stenosis” as used herein, refers to narrowing or stricture of a hollow passage (e.g. a duct or canal) in the body.

The phrase “vascular stenosis” refers to occlusion or narrowing of blood vessels. Vascular stenosis often results from fatty deposit (as in the case of atherosclerosis) or excessive migration and proliferation of vascular smooth muscle cells and endothelial cells. Arteries are particularly susceptible to stenosis. Thus, the term “stenosis” as used herein specifically includes initial stenosis and restenosis.

The term “restenosis” refers to recurrence of stenosis after treatment of initial stenosis with apparent success. For example, in the context of vascular stenosis, restenosis refers to the reoccurrence of vascular stenosis after it has been treated with apparent success, e.g. by removal of fatty deposit by angioplasty (e.g. percutaneous transluminal coronary angioplasty), direction coronary atherectomy or stent. One of the contributing factors in restenosis is intimal hyperplasia. The term “intimal hyperplasia”, used interchangeably with “neointimal hyperplasia” and “neointima formation”, refers to thickening of the inner most layer of blood vessels, intima, as a consequence of excessive proliferation and migration of vascular smooth muscle cells and endothelial cells. The various changes taking place during restenosis are often collectively referred to as “vascular wall remodeling”. The terms “balloon angioplasty” and “percutaneous transluminal coronary angioplasty” (PTCA) are often used interchangeably, and refer to a non-surgical catheter-based treatment for removal of plaque from the coronary artery. Stenosis or restenosis often lead to hypertension as a result of increased resistance to blood flow.

The term “hypertension” refers to abnormally high blood pressure, beyond the upper value of the normal range.

The term “polyp” refers to a mass of tissue that bulges or projects outward or upward from the normal surface level, thereby being macroscopically visible as a hemispheroidal, speroidal, or irregular mound-like structure growing from a relatively broad base or a slender stalk. Examples include colon, rectal and nasal polyps.

The term “fibroadenoma” refers to a benign neoplasm derived from glandular epithelium, in which there is a conspicuous stroma of proliferating fibroblasts and connective tissue elements. This commonly occurs in breast tissue.

The term “asthma” refers to a medical condition which results in difficulty in breathing. Bronchial asthma refers to a condition of the lungs in which there is widespread narrowing of airways, which may be due to contraction (spasm) of smooth muscle, edema of the mucosa, or mucus in the lumen of the bronchi and bronchioles. The term “bronchitis” refers to inflammation of the mucous membrane of the bronchial tubes.

According to some embodiments of the present invention, the compounds presented herein can be used to treat non-malignant proliferative disease or disorder such as psoriasis, endometriosis, scleroderma, a vascular disease, colon polyps, fibroadenoma and a respiratory disease.

As used herein, the phrase “therapeutically effective amount” describes an amount of the compound being administered which will relieve to some extent one or more of the symptoms of the condition being treated.

As demonstrated in the examples section that follows, an exemplary therapeutically effective amount of the compounds of the present invention ranges between about 0.1 mg/kg body and about 100 mg/kg body.

In any of the methods and uses described herein, the cannabidiol derivative compounds of the present embodiments can be utilized either per se or, according to some embodiments, as a part of a pharmaceutical composition that further comprises a pharmaceutically acceptable carrier.

Thus, according to additional aspects of the present invention, there is provided a pharmaceutical composition, which comprises one or more compounds having general Formula I, as defined hereinabove, and a pharmaceutically acceptable carrier.

According to some embodiments of the present invention, there is provided a pharmaceutical composition, which comprises one or more of any one of the compounds presented in Table 1 which is presented in the Examples section that follows hereinbelow, and a pharmaceutically acceptable carrier, as well as any enantiomers, hydrates, solvates, prodrugs or any pharmaceutically acceptable salts thereof, as defined hereinabove.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the compounds into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of a proliferative disease or disorder, as is detailed hereinabove.

Thus, according to an embodiment of the present invention, the pharmaceutical composition of the present invention is being packaged in a packaging material and identified in print, in or on the packaging material, for use in the treatment of a proliferative disease or disorder, as is defined hereinabove.

According to further embodiments of the any of the methods, uses and compositions presented herein, the compounds of the present invention can be combined with other active ingredients which are commonly used to treat cell proliferation-associated diseases and disorders.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions; illustrate the invention in a non limiting fashion.

### Example 1

### Chemical Syntheses

Materials and Methods:

All chemical reagents were purchased from Sigma-Aldrich.

Organic solvents were purchased from Bio-Lab Ltd., Israel.

Cannabinoids were extracted from Cannabis sativa plant as previously described [17].

Oxidation of a Cannabinoid Derivative to a Quinonoid Derivative—A General Procedure:

A cannabinoid having a general Formula I′ is oxidized to a quinonoid derivative having general Formula I using bis[trifluoroacetoxy]iodobenzene (BTIB), as illustrated in Scheme 2 below.

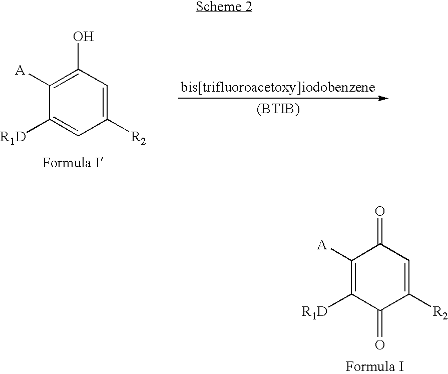

A solution of BTIB (0.3 mmol) in acetonitrile/water (6:1, 0.7 ml) is added dropwise to a solution of the cannabinoid (0.15 mmol) in acetonitrile/water (6:1, 0.7 ml). The reaction mixture is stirred at room temperature for 15 minutes, neutralized with aqueous NaHCO₃ saturated solution and extracted with diethyl ether. Thereafter, the organic layer is washed with water, dried over MgSO₄, concentrated under reduced pressure and purified by column chromatography to afford the quinonoid.

### Preparation of ethyl 2-(2-((6R)-3-methyl-6-(prop-1-en-2-yl)cyclohex-2-enyl)-3,6-dioxo-5-pentcyclohexa-1,4-dienyloxy)acetate (HU-701)

Ethyl 2-(3-hydroxy-2-((6R)-3-methyl-6-(prop-1-en-2-yl)cyclohex-2-enyl)-5-pentylphenoxy)acetate was oxidized to ethyl 2-(2-((6R)-3-methyl-6-(prop-1-en-2-yl)cyclohex-2-enyl)-3,6-dioxo-5-pentylcyclohexa-1,4-dienyloxy)acetate (HU-701) using bis[trifluoroacetoxy]iodobenzene (BTIB), according to the general procedure presented above and as illustrated in Scheme 2 above.

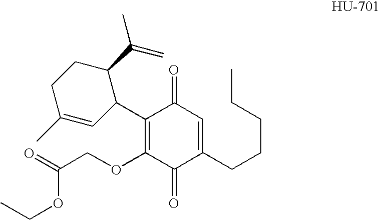

A solution of BTIB (0.3 mmol) in acetonitrile/water (6:1, 0.7 ml) was added dropwise to a solution of ethyl 2-(3-hydroxy-2-((6R)-3-methyl-6-(prop-1-en-2-yl)cyclohex-2-enyl)-5-pentylphenoxy)acetate (0.15 mmol) in acetonitrile/water (6:1, 0.7 ml). The reaction mixture was stirred at room temperature for 15 minutes, neutralized with aqueous NaHCO₃ saturated solution and extracted with diethyl ether. Thereafter, the organic layer was washed with water, dried over MgSO₄ and concentrated under reduced pressure and purified by column chromatography to afford ethyl 2-(2-((6R)-3-methyl-6-(prop-1-en-2-yl)cyclohex-2-enyl)-3,6-dioxo-5-pentylcyclohexa-1,4-dienyloxy)acetate (HU-701) at a 20% yield.

### Preparation of ethyl 2-(2-((6R)-3-methyl-6-(prop-1-en-2-yl)cyclohex-2-enyl)-3,6-dioxo-5-pentylcyclohexa-1,4-dienyloxy)acetic acid (HU-702)

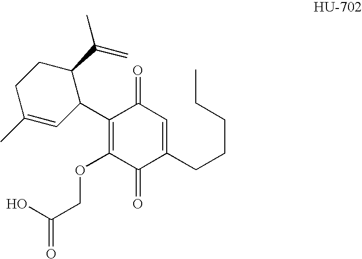

Oxidation of 2-(3-hydroxy-2-((6R)-3-methyl-6-(prop-1-en-2-yl)cyclohex-2-enyl)-5-pentylphenoxy)acetic acid to 2-(2-((6R)-3-methyl-6-(prop-1-en-2-yl)cyclohex-2-enyl)-3,6-dioxo-5-pentylcyclohexa-1,4-dienyloxy)acetic acid (HU-702) at 20% yield using bis[trifluoroacetoxy]iodobenzene (BTIB) was carried out according to the general procedure presented above and as illustrated in Scheme 2 above.

Other exemplary cannabinoid-derived quinonoid derivative compounds according to embodiments of the present invention, prepared according to the general to procedure presented above and as illustrated in Scheme 2 starting from the corresponding cannabinoid derivative compounds, are presented in Table 1 hereinbelow.

TABLE 1
Compound name and

| code | Structure | Formula I |
|---|---|---|
| ethyl 2-(2-((6R-3- methyl-6-(prop-1-en-2- yl)cyclohex-2-enyl)-3,6- dioxo-5- pentylcyclohexa-1,4- dienyloxy)acetate (HU-701) | 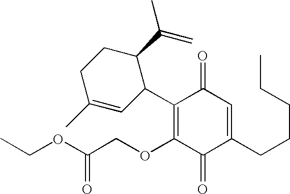 | D = O; A = 3-methyl-6-(prop-l-en- 2-yl)cyclohex-2-enyl; R₁ = ethoxy-2-oxo-ethane- l-yl; R₃ = 1-pentyl |
| 2-(2-((6R)-3-methyl-6- (prop-l-en-2- yl)cyclohex-2-enyl)-3,6- dioxo-5- pentylcyclohexa-1,4- dienyloxy)acetic acid (HU-702) | 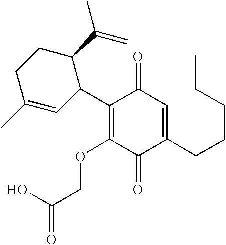 | D = O; A = 3-methyl-6-(prop-l-en- 2-yl)cyclohex-2-enyl; R₁ = 2-yl-acetic acid; R₃ = 1-pentyl |
| 3-(2-hydroxyethoxy)-2- ((6R)-3-methyl-6-(prop- 1-en-2-yl)cyclohex-2- enyl)-5- pentylcyclohexa-2,5- diene-1,4-dione (HU-703) | 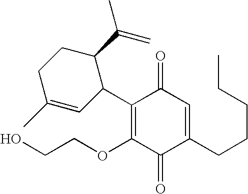 | D = O; A = 3-methyl-6-(prop-1-en- 2-yl)cyclohex-2-enyl; R₁ = ethanol-2-yl; R₃ = 1-pentyl |
| 3-(2-aminoethoxy)-2- ((6R)-3-methyl-6-(prop- 1-en-2-yl)cyclohex-2- enyl)-5- pentylcyclohexa-2,5- diene-1,4-dione (HU-704) | 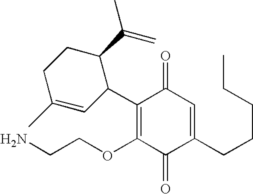 | D = O; A = 3-methyl-6-(prop-1-en- 2-yl)cyclohex-2-enyl; R₁ = ethanamine-2-yl; R₃ = 1-pentyl |
| 3-hydroxy-2-(1- methylpiperidin-4-yl)-5- pentylcyclohexa-2,5- diene-1,4-dione (HU-705) | 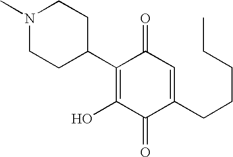 | D = O; A = 3-methyl-6-(prop-1-en- 2-yl)cyclohex-2-enyl; R₁ = hydrogen R₃ = 1-pentyl |

### Example 2

### Biological Activity

The anti-proliferative activity of exemplary cannabinoid-derived quinonoid derivative compounds, according to some embodiments of the present invention, was tested as presented below.

Preparation of Jurkat Cells for Anti Proliferative Activity Assays:

Jurkat cells were suspended in RPMI 1640 medium, supplemented with 20% heat-inactivated fetal calf serum (H-I FCS), 2 mM L-glutamine, 100 U/ml penicillin, and 0.01 mg/ml streptomycin at 37° C. in a 5% CO₂ humidified atmosphere. HT-29 cells were suspended in RPMI 1640 medium, supplemented with 10% H-I FCS, 2 mM L-glutamine, 100 U/ml penicillin, and 0.01 mg/ml streptomycin at 37° C. in a 5% CO₂ humidified atmosphere.

Cell Proliferation Test:

Aliquots (200 μl) of suspensions of cancer cells were dispensed into wells of 96-well tissue culture plates at densities of 0.02×106 cells/well. Various concentrations of quinonoid derivative compounds were introduced into the wells, and their efficacy was tested three days after initiation of the cultures, using the 3-(4,5-dimethylthiazol-2-yl)-2,5-diphenyltetrazolium bromide (MTT) assay.

The principle of this assay is that cells which survive following exposure to various compounds can reduce MTT to a dark-colored formazan, while dead cells are incapable of doing so. The assay was performed as described previously [18-20].

In each MTT assay every concentration of the cytotoxic substance was tested in five replicates in microplate wells. Assays with every cell line were carried out in two to three repeated experiments. The inhibitory effect of various compounds was calculated as percentage inhibition in comparison with the values obtained in untreated wells to which vehicle (0.5% ethanol) was added.

### Results

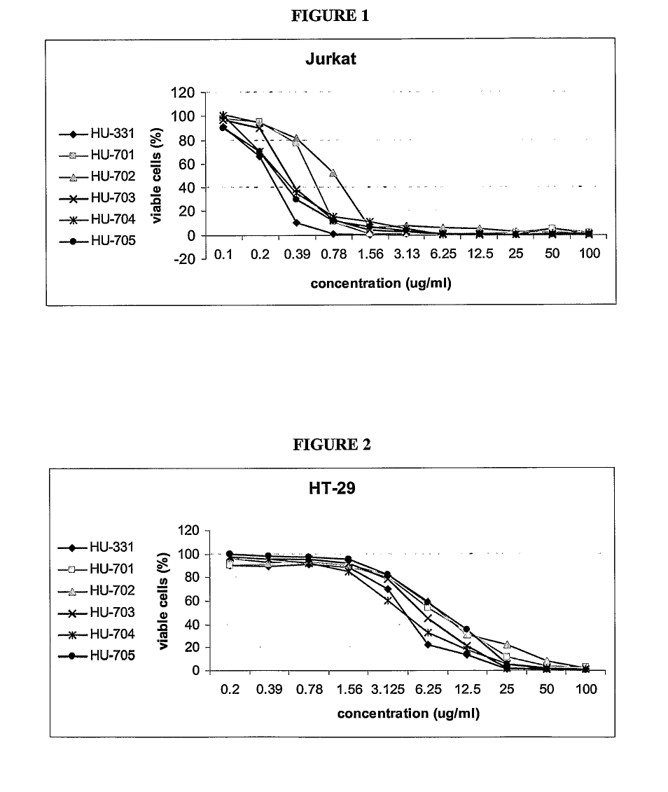

Several exemplary compounds according to some embodiments of the present invention, namely HU-701, HU-702, HU-703, HU-704 and HU-705, were tested for inhibiting growth of human cancer cell lines, and compared to HU-331, a known cannabinoid-based anticancer agent. The results of the biological activity assays are presented in FIGS. 1 and 2.

FIG. 1 presents the results of an in-vitro cell proliferation assay on Jurkat human lymphoma cell line.

FIG. 2 presents the results of an in-vitro cell proliferation assay of human colon carcinoma HT-29 cell line.

As can be seen in FIGS. 1 and 2, all the tested compound, designed according to some embodiments of the present invention, inhibited human cancer cell lines growth. As can further be seen in both FIGS. 1 and 2, the compounds presented herein showed anti-proliferative activity superior to that of HU-331.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

### Other References are Cited in the Text

1. Thomas, X., et al., Ann Hematol, 2002. 81(9): p. 504-7.
2. Zucchi, R., et al., Curr Med Chem Anticancer Agents, 2003.3(2): p. 151-71.
3. Aiello, A., et al., J Med Chem, 2005. 48(9): p. 3410-6.
4. Razdan, R. K., Pharmacol Rev, 1986. 38(2): p. 75-149.
5. Mechoulam, R., et al., Prog Med Chem, 1998. 35: p. 199-243.
6. Barth, F., et al., Curr Med Chem, 1999. 6(8): p. 745-55.
7. Mechoulam, R., et al., Tetrahedron 1968. 24(16): p. 5615-24.
8. Watanabe, K., et al., J Pharmacobiodyn, 1991. 14(7): p. 421-7.
9. Bornheim, L. M., et al., Chem Res Toxicol, 1998. 11(10): p. 1209-16.
10. Usami, N., et al., Res. Commun. Alcohol and Substances of Abuse, 1999. 20(1 and 2): p. 53-68.
11. Kogan, N. M., et al., J Med Chem, 2004. 47(15): p. 3800-6.
12. Danheiser, R. L., et al., Science of Synthesis, 2006. 23: p. 493-568.
13. Kim, S. H., et al., Science of Synthesis, 2006. 28: p. 53-69.
14. Kogan, N. M., et al., Mol Pharmacol, 2006. 70(1): p. 51-9.
15. Kogan, N. M., et al., J Pharmacol Exp Ther, 2007. 322(2): p. 646-53.
16. Peters, M., et al., Expert Opin Investig Drugs, 2007. 16(9): p. 1405-13.
17. Gaoni, Y., et al., J Am Chem Soc 1964. 86: p. 1646-1647.
18. Carmichael, J., et al., Cancer Res, 1987. 47(4): p. 936-42.
19. Rubinstein, L. V., et al., J Natl Cancer Inst, 1990. 82(13): p. 1113-8.
20. Rubnov, S., et al., J Nat Prod, 2001. 64(7): p. 993-6.

1.-34. (canceled)

35. A compound having general Formula I: 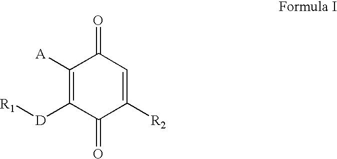
an enantiomer, a hydrate, a solvate or a pharmaceutically acceptable salt thereof;
wherein:
A is selected from the group consisting of an unsubstituted or substituted cycloalkyl, an unsubstituted or substituted heteroalicyclic, an unsubstituted or substituted aryl and a substituted heteroaryl;
R₁ is selected from the group consisting of hydrogen and an unsubstituted or substituted, branched or linear alkyl having from 1 to 10 carbon atoms; and
R₂ is selected from the group consisting of an unsubstituted or substituted, branched or linear alkyl having from 1 to 10 carbon atoms, an alkoxy and an aryloxy;
D is selected from the group consisting of NR₃, O and S; and
R₃ is an unsubstituted or substituted, branched or linear alkyl having from 1 to 10 carbon atoms;
excluding compounds of formula I wherein A is cycloalkyl or aryl, D is O, R₁ is a hydrogen or an unsubstituted branched or linear alkyl having 1 to 5 carbon.

36. The compound of claim 35, wherein D is O.

37. The compound of claim 35, wherein D is O and A is selected from the group consisting of an unsubstituted or substituted cycloalkyl, an unsubstituted or substituted heteroalicyclic and substituted heteroaryl.

38. The compound of claim 35, wherein D is O and A is an unsubstituted or substituted heteroalicyclic.

39. The compound of claim 35, wherein D is O; A is an unsubstituted or substituted heteroalicyclic; and R₂ is selected from the group consisting of pentyl and dimethyl-heptyl.

40. The compound of claim 35, wherein D is O; A is an unsubstituted or substituted heteroalicyclic; R₂ is selected from the group consisting of pentyl and dimethyl-heptyl and R₁ is hydrogen.

41. The compound of claim 35, wherein D is O; A is 1-methylpiperidin-4-yl; R₂ is selected from the group consisting of pentyl and dimethyl-heptyl and R₁ is hydrogen.

42. The compound of claim 35, wherein D is O and A is an unsubstituted or substituted cycloalkyl, selected from the group consisting of a monocyclic unsubstituted or substituted cycloalkyl and a bicyclic unsubstituted or substituted cycloalkyl.

43. The compound of claim 42, wherein the bicyclic unsubstituted or substituted cycloalkyl is an unsubstituted or substituted pinene.

44. The compound of claim 42, wherein the monocyclic unsubstituted or substituted cycloalkyl has general: 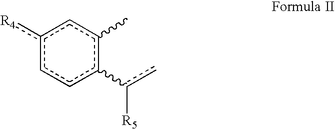
wherein:
R₄ and R₅ are each independently selected from the group consisting of hydrogen, alkyl, haloalkyl, halo, hydroxyl, alkoxy, carboxyl, carbonyl, formyl, acetyl and amine;
whereas:
a dashed line is a single or double bond; and
a wavy line is a bond having an R or an S stereo-configuration.

45. The compound of claim 35, wherein R₁ is selected from the group consisting of an unsubstituted or substituted, branched or linear alkyl having from 6 to 10 carbon atoms and a substituted, branched or linear alkyl having from 1 to 10 carbon atoms.

46. The compound of claim 35, wherein R₁ is a substituted, branched or linear alkyl having from 1 to 5 carbon atoms.

47. The compound of claim 35, wherein A is 3-methyl-6-(prop-1-en-2-yl)cyclohex-2-enyl.

48. The compound of claim 35, wherein A is 3-methyl-6-(prop-1-en-2-yl)cyclohex-2-enyl and R₁ is selected from the group consisting of 2-yl-acetic acid, ethyl 2-yl-acetate, ethoxy-2-oxo-ethane-1-yl, ethanol-2-yl, ethanamine-2-yl, N-Boc-ethanamine-2-yl, N-Fmoc-ethanamine-2-yl, 3-morpholinopropanoyl, and acetonitrile-2-yl.

49. The compound of claim 35, wherein A is 3-methyl-6-(prop-1-en-2-yl)cyclohex-2-enyl and R₁ is selected from the group consisting of ethoxy-2-oxo-ethane-1-yl, 2-yl-acetic acid, ethanol-2-yl, and ethanamine-2-yl.

50. The compound of claim 35, wherein A is 3-methyl-6-(prop-1-en-2-yl)cyclohex-2-enyl; R₁ is selected from the group consisting of ethoxy-2-oxo-ethane-1-yl, 2-yl-acetic acid, ethanol-2-yl and ethanamine-2-yl; and R₂ is selected from the group consisting of pentyl and dimethyl-heptyl.

51. The compound of claim 35, wherein A is 3-methyl-6-(prop-1-en-2-yl)cyclohex-2-enyl; R₁ is selected from the group consisting of ethoxy-2-oxo-ethane-1-yl, 2-yl-acetic acid, ethanol-2-yl, and ethanamine-2-yl; and R₂ is 1-pentyl.

52. The compound of claim 35, wherein R₂ is selected from the group consisting of pentyl and dimethyl-heptyl.

53. The compound of claim 35, having an anti-proliferative activity.

54. A compound selected from the group consisting of:
ethyl 2-(2-((6R)-3-methyl-6-(prop-1-en-2-yl)cyclohex-2-enyl)-3,6-dioxo-5-pentylcyclohexa-1,4-dienyloxy)acetate (HU-701);
2-(2-((6R)-3-methyl-6-(prop-1-en-2-yl)cyclohex-2-enyl)-3,6-dioxo-5-pentylcyclohexa-1,4-dienyloxy)acetic acid (HU-702);
3-(2-hydroxyethoxy)-2-((6R)-3-methyl-6-(prop-1-en-2-yl)cyclohex-2-enyl)-5-pentylcyclohexa-2,5-diene-1,4-dione (HU-703);
3-(2-aminoethoxy)-2-((6R)-3-methyl-6-(prop-1-en-2-yl)cyclohex-2-enyl)-5-pentylcyclohexa-2,5-diene-1,4-dione (HU-704);
3-hydroxy-2-(1-methylpiperidin-4-yl)-5-pentylcyclohexa-2,5-diene-1,4-dione (HU-705); and
any enantiomer, hydrate, solvate or pharmaceutically acceptable salt thereof.

55. The compound of claim 54, having an anti-proliferative activity.

56. A pharmaceutical composition comprising as an active ingredient the compound of claim 35.

57. The pharmaceutical composition of claim 56, being packaged in a packaging material and identified in print, in or on the packaging material, for use in the treatment of a proliferative disease or disorder.

58. A method of treating a proliferative disease or disorder, the method comprising administering to a patient in need thereof a therapeutically effective amount of the compound of claims 35.

59. The method according to claim 58, wherein the proliferative disease or disorder is selected from the group consisting of a malignant proliferative disease or disorder, a non-malignant proliferative disease or disorder, an inherent proliferative disease or disorder, and an acquired proliferative disease or disorder.
